(19) United States
(12) Patent Application Publication
(10) Pub. No.: US 2013/0217639 A1
(43) Pub. Date: Aug. 22, 2013

(54) COMPOUND FOR ACTIVATING NATURAL KILLER T CELLS AND PREPARING METHOD THEREOF

(71) Applicants: Chung-Shan Yu, Taipei City (TW); Li-Wu Chiang, Taipei City (TW); Kai-Hsiang Chang, Taipei City (TW); Kuo-Yen Liao, Taichung City (TW); Wen-Chin Su, Changhua County (TW); Yin-Cheng Huang, New Taipei City (TW)

(72) Inventors: Chung-Shan Yu, Taipei City (TW); Li-Wu Chiang, Taipei City (TW); Kai-Hsiang Chang, Taipei City (TW); Kuo-Yen Liao, Taichung City (TW); Wen-Chin Su, Changhua County (TW); Yin-Cheng Huang, New Taipei City (TW)

(73) Assignee: NATIONAL TSING HUA UNIVERSITY, Hsinchu (TW)

(21) Appl. No.: 13/402,342

(22) Filed: Feb. 22, 2012

(51) Int. Cl. A61K 31/7028; C40B 30/00; C07H 15/22

(52) U.S. Cl. 514/25; 536/17.9; 506/7

(57) ABSTRACT

The present invention provides a compound selected from compound of formula (I), pharmaceutically acceptable salts of compound of formula (I), and pharmaceutically acceptable prodrugs of compound of formula (I),

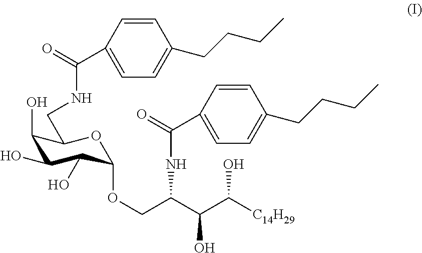

and a composition comprising the compound of the above and a pharmaceutically acceptable excipient.

The present invention also provides a method for preparing a compound that activates natural killer T cells.

### FIELD OF THE INVENTION

The present invention relates to a compound selected from compound of formula (I)

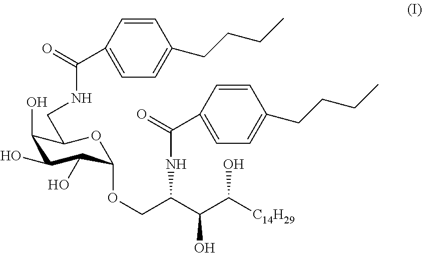

and a method for preparing a compound that activates natural killer T cells.

### BACKGROUND OF THE INVENTION

α-Galactosyl ceramide (α-GalCer), also called KRN7000 (FIG. 1), attracts great attention due to its biological significances such as antitumor effects (Singh, R.; Sharma, M.; Joshi, P.; Rawat, D. S. Anti-Cancer Agents in Medicinal Chemistry, 2008, 8, 603). The complex formed between α-GalCer and CDld can stimulate invariant natural killer T cells (iNKT) to secret cytokines of diverse cytokines such as Thl and Th2 cytokines (Kronenberg, M.; Gapin, L. PNAS 2007, 104, 5713). It is believed that the release of Thl cytokines may contribute to antitumor and antimicrobial functions (Chang, Y.-J.; Huang, J.-R.; Tsai, Y.-C.; Hung, J.-T.; Wu, D.; Fujio, M.; Wong, C.-H.; Yu, A. L. Proc. Natl. Acad. Sci. U.S.A. 2007, 104, 10299). Various analogs of α-GalCer have thus been developed to induce cytokine bias, for example, modification on glycosidic bonding with sulfur (Dere, R. T.; Zhu, X. Org. Lett. 2008, 10, 4641), nitrogen (Harrak, Y.; Barra, C. M.; Bedia, C.; Delgado, A.; Castano, A. R.; Llebaria, A. ChemMedChem 2009, 4, 1608), carbon (Lu, X.; Song, L.; Metelitsa, L. S.; Bittman, R. ChemBioChem 2006, 7, 1750) or oxime (Chen, W.; Xia, C.; Cai, L.; Wang, P. G. Bioorg. Med. Chem. Lett. 2010, 20, 3859) and substituent modification on acyl chain (Fujio, M.; Wu, D.; Garcia-Navarro, R.; Ho, D. D.; Tsuji, M.; Wong, C. H. J. Am. Chem. Soc. 2006, 128, 9022). For diversifying the compound pools, library approach could provide a straightforward manner. The recent development of a -GalCer libraries including solution-phase-synthesis approach by Wong (Fujio, M.; Wu, D.; Garcia-Navarro, R.; Ho, D. D.; Tsuji, M.; Wong, C. H. J. Am. Chem. Soc. 2006, 128, 9022) and solid phase synthesis approach by Howell (Li, Q.; Ndonye, R. M.; Illarionov, P. A.; Yu, K. 0. A.; Jerud, E. S.; Diaz, K.; Bricard, G.; Porcelli, S. A.; Besra, G. S.; Chang, Y.-T.; Howell, A. R. J. Comb. Chem. 2007, 9, 1084) has generated a number of compounds. Both purity and identity can be established as well. For example, Howell's elegant synthesis using solid phase approach could deliver the amide product in a straightforward manner. Wong et al had also ingeniously discovered that the bioactivity pattern could be biased by introducing aromatic substituents on the acy chain.

Numerous approaches to structural modification the sugar head and truncation of the sphingosine backbone or acy chain as well as incorporation of unsaturation in the acyl chain have generated some bioactive compound leads. For example, some of the truncated compounds are TH2-biased pathway, whereas only rarer cases could lead toward TH1-biased pathway. α-GalCer analogs such as C-modified glycosidic linkage have been shown to possess this feature, probably due to its inertness to metabolic cleavage of glycosidic bond. Hence, amide bond with reasonable inertness might provide an alternative to glycosidic bond.

### BRIEF DESCRIPTION OF THE DRAWINGS

FIG. 1 shows that α-GalCer and structural analogue with stable glycosidic bond may resist metabolic cleavage.

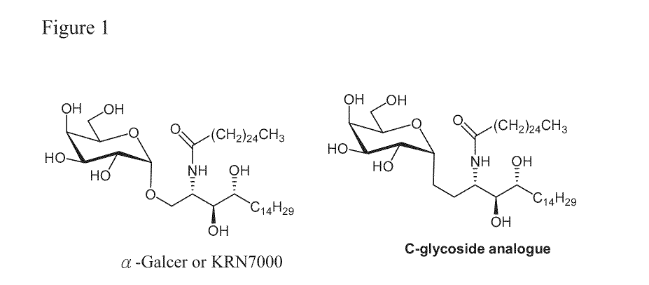

FIG. 2 shows that structural modification using amide may resist metabolic cleavage. The library moieties to be prepared may modify the cytotoxicity as well as immuno stimulating effect.

FIG. 3 shows donors and acceptors used for preparing glycosylated products.

FIG. 4 shows potencies of analogs 18 and 19 for stimulation of human Vα24+/V−β11+ NKT cell populations. Peripheral blood mononuclear cells (PBMC) from a normal healthy donor were incubated with each individual compound at a final concentration of 100 nM. After 14 days of culture, NKT cell frequencies were determined by flow cytometry. NKT cell frequencies were defined as the percentage of Vα24+/V−β11+ cells among gated lymphocytes in the upper right (UR) corner for each case. Shown here are the profiles of PBMC harvested from 14-day cultures containing (a) vehicle alone (DMSO, UN), or (b) 100 nM of α-GalCer (KS), (c) analog 18 (DABB), or (d) analog 19 (DAGBB), as indicated.

### SUMMARY OF THE INVENTION

The present invention relates to a compound selected from compound of formula (I), pharmaceutically acceptable salts of compound of formula (I), and pharmaceutically acceptable prodrugs of compound of formula (I),

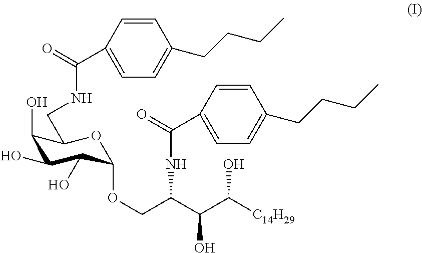

and a composition comprising the compound of the above and a pharmaceutically acceptable excipient.

The present invention also relates to a method for preparing a compound that activates natural killer T cells, comprising:

establishing a library by coupling a compound of formula (II) with at least 44 carboxylic acids;

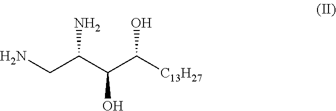

using MTT assay to screen for the cytotoxicities of these amide product mixtures of the library;

analyzing the amide product mixtures showing less cytotoxicities by ESI-MS; wherein the amide product mixtures showing less cytotoxicities are the amide product mixtures with viability at least 10% higher than average viability in the MTT assay;

resynthesizing the amide product showing significant MS signals in its galactosyl ceramide form; and

validating the activity of the galactosyl ceramide by flowcytometry.

### DETAILED DESCRIPTION OF THE INVENTION

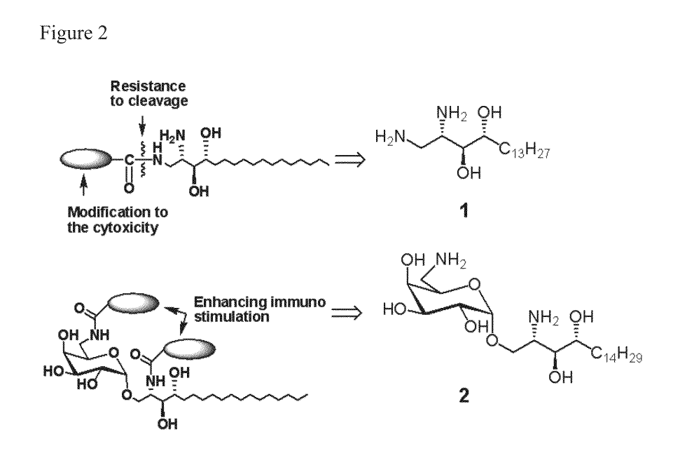

In the present invention, an amide library derived from 1-amino phytosphingosine analogs 1 with variation of acyl groups is prepared and screened to find which structural features had moderate cytotoxicities. With such a structural type in hand, compounds that incoprporated this acyl group into α-galactosyl sphingosine 2 at 6-amino of sugar and (or) 2-amino of sphingoid base are evaluated for its immunostimulating potency. The concept for the design of the synthesis and screening of the present invention is outlined in FIG. 2.

The present invention comprises three parts: 1) the preparation of a novel 1,2-diamino phytosphingosine; 2) preparation of 6-azido thio galactoside with ester-type and ether-type donors for obtaining glycosylated compounds in both acceptable yield and stereoselectivity; and 3) the in-situ screening of the cellular cytotoxicity and the validation of the purified compounds.

In the present invention, analogs of a-GalCer are synthesized for stimulatory effect to human iNKT cells. 1,2-diamino phytosphingosine and 6-aminogalactosyl phytosphingosine are prepared with 61% and 40% yield, respectively. Glycosylation using benzoyl-protected lipid resultes in better a-selectivity but less yield than that by benzyl moieties. Screening the amide libraries with 105 carboxylic acids shows that 4-butyl benzoic acid-derived product exerts less cytotoxicity. These analogs are purified for validation of immunological potencies and α-GalCer analog 19 but not analog 18 stimulats human iNKT cell population.

Therefore, the present invention provides a compound selected from compound of formula (I), pharmaceutically acceptable salts of compound of formula (I), and pharmaceutically acceptable prodrugs of compound of formula (I)

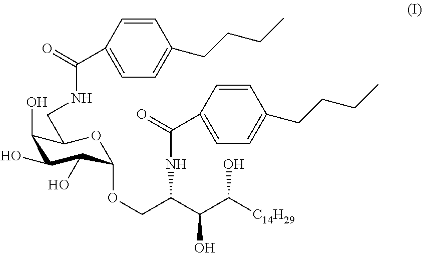

The present invention also provides a composition comprising the compound of the above and a pharmaceutically acceptable excipient.

The present invention further provides a method for preparing a compound that activates natural killer T cells, comprising:

establishing a library by coupling a compound of formula (II) with at least 44 carboxylic acids;

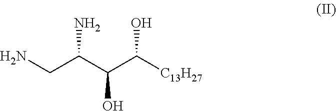

using MTT assay to screen for the cytotoxicities of these amide product mixtures of the library;

analyzing the amide product mixtures showing less cytotoxicities by ESI-MS; wherein the amide product mixtures showing less cytotoxicities are the amide product mixtures with viability at least 10% higher than average viability in the MTT assay;

resynthesizing the amide product showing significant MS signals in its galactosyl ceramide form; and

validating the activity of the galactosyl ceramide by flowcytometry.

The “significant MS signals” are defined as signals which can be identified directly in the MS without further signal amplification.

### EXAMPLES

The examples below are non-limiting and are merely representative of various aspects and features of the present invention.

### Example 1

Both commercial available and well-protected phytosphingosines from Garner aldehyde were used as starting materials to prepare the target compounds 2 and 1 (Chiang, L.-W.; Pan, S.-D.; Lo, J.-M.; Yu, C.-S. Chin. J. Chem. 2009, 27, 2296). Thus, the current synthetic strategy attempted to use the azide as masked functionality for both phytosphingosine base and sugar portion. Introduction of the azido group under a copper-catalyzed condition was reacted mildly (Scheme 1). The subsequent introduction of the triflate did not lead to the desired product 5 but only the cyclized analog of 2-epi jaspin B. 6 (In rare cases, it was able to isolate the triflate 5. The fragment peaks appeared in ESI-MS spectrum such as 479.3 amu (27%), 493.4 amu (2.4%) and 595.6 amu (2.4%) indicated that the instability of triflate could lead to a number of intermediates. Satisfactory ¹H-NMR spectra were, however, not available due to the complex patterns.), a potential anti-cancer compound reported recently (Enders, D.; Terteryan, V.; Palecek, J. Synthesis, 2008, 2278).

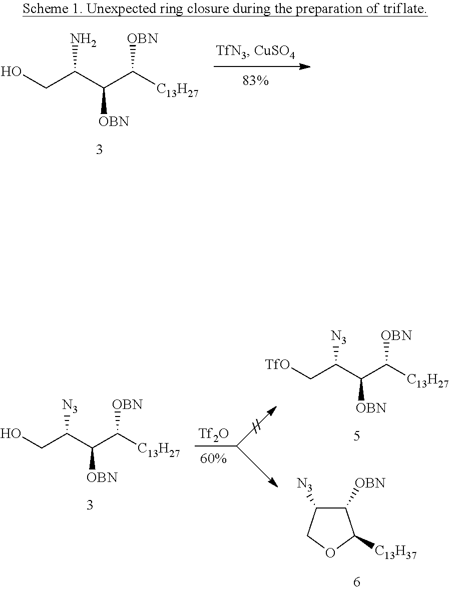

Introduction of the tosyl group by using tosyl chloride underwent smoothly without encountering the problem of ring closure (Scheme 2). The subsequent nucleophilic attack by azide afforded the desired diazido compound 8 in 80% yield accompanied with the cyclized 2-epi jaspin B analog 6. The following reduction reaction using BCl₃ gave the desired diamino phytosphingosine analog 1 in quantitative yield. Interestingly, when using less equivalents of BCl₃ (5 eq), the primary azide was selectively reduced to afford monoamino compound 9.

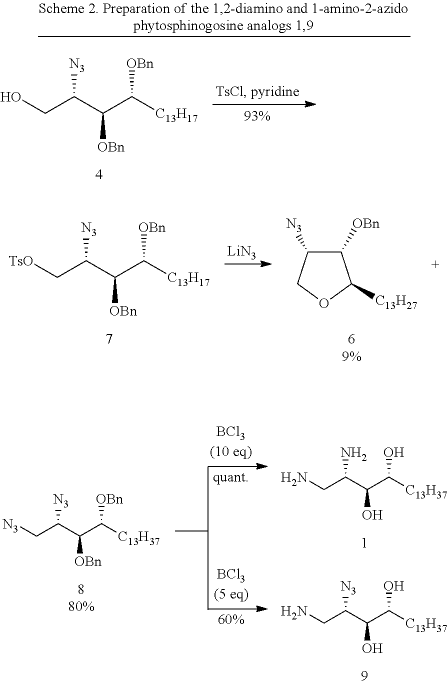

As observed in the ¹H-NMR for the diamino compound 1, the two broad peaks at 8.26 and 8.47 ppm indicated the presence of ammonium complexes. Although both ¹H- and ¹³C-NMR spectra for the slightly-light-brown sample were satisfactory, it could be purified to white solid through ion exchange (OH⁻) resin. The ESI-MS spectrum implied the improvement in purity (The chemical shifts of protons from Cl to C4 in ¹H-NMR were slightly upfield. Interestingly, the two ammonium protons were no longer observable between 8 and 9 ppm, indicating the presence of neutral amine rather than the ammonium ion.). Furthermore, the ESI-MS spectra also indicated the different fragmentation patterns between the two complexes (The protons of the ammonium complex with HCl could be observed in ¹H-NMR. By contrast, no peaks could be found in ESI-MS. HCl is easier evaporated during electrospraying step and thereby only the neutral amino form emerged as the base peak, 303.4 (m/z). In contrast, a substantial amount of the ammonium hydroxide form would be preserved during ESI thereby appearing as the base peak. The patterns of peak clustering around 389.3 (m/z) implied the presence of chloro-containing molecular ion.).

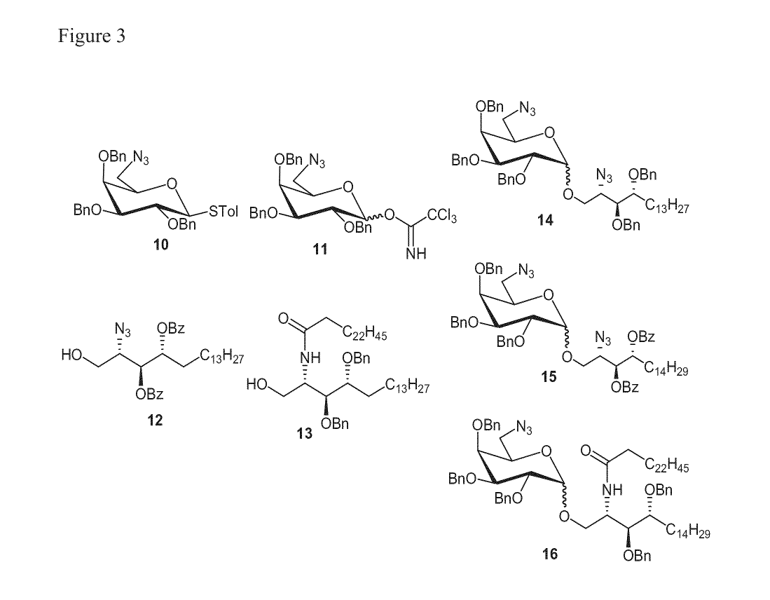

For synthesizing the galactosyl phytosphingosine, the 6-azido galactosyl thioglycoside 10 was used as a donor (FIG. 3) (Jacobsson, M.; Malmberg, J.; Ellervik, U. Carbohydr. Res. 2006, 341, 1266). Glycosylation using both ether-protected donor and acceptor, the so-called “armed glycosylation”, could deliver products in high yield but diminished stereoselectivity (Table 1, entry 1) (Mydock, L. K.; Demchenco, A. V. Org. Lett. 2008, 10, 2103). On the other hand, glycosylation using benzoyl-protected sphingosine 12, a disarmed acceptor, could provide products 5 in fair yield but slightly improved selectivity (entry 2). This was attributed to an oxocarbenium ion preformed before the nucleophilic attack by lipid (Xia, C. F.; Yao, Q. J.; Schumann, J.; Rossy, E.; Chen, W. L.; Zhu, L. Z.; Zhang, W. P.; De Libero, G.; Wang, P. G. Bioorg. Med. Chem. Lett 2006, 16, 2195). When a benzyl-protected ceramide 13 was used as an acceptor, only very limited amounts of the glycosylated products 16 were obtained (entry 3) (Anal. C₈₃H₁₂₄N₄O₈, M (calcd.)=1304.9 (m/z); ESI+Q-TOF: M=1304.8 (m/z), [M+Na]⁺=1327.8 (8.7%), 1328.6 (7.8%), 1329.5 (3.6%), approximately equivalent to the calculated isotopic ratio (100% : 91.5%: 43.0%)). The poor yield could be due to the neighboring amido hydrogen donor by decreasing the nucleophilicity of primary alcohol (Enders, D.; Terteryan, V.; Palecek, J. Synthesis, 2008, 2278). It has been reported that imidate as a donor could achieve excellent yield and a-stereoselectivity in glycosylation (Du, W.; Kulkarni, S. S.; Gervay-Hague, J. Chem. Comm. 2007, 2336). By adopting the similar condition, only the undesired silylated alcohol was obtained, whereas the imidate was consumed (entry 4) (¹H-NMR (300 MHz, CDCl₃): 6 0.10 (s, 9H, CH₃), 0.90 (t, 3H, CH_{3(aliphatic)}), 1.15-1.50 (m, 24 H, H₄)1.80-2.00 (m, 2H, H_{aliphatic}), 3.75-4.00 (m, 3H), 5.46-5.56 (m, 2H, H₃, H₄), 7.40-7.50 (m, 4H, ArH), 7.53-7.63 (m, 2H, ArH), 7.98-8.00 (m, 4H, ArH)). A similar result was obtained when using ceramide 13 as an acceptor (entry 5); and the problem might be caused by the discrepancy in reactivity between acceptor and donor.

TABLE 1
Glycosylation between sphingosine analogs 4, 12, 13 and 6-azido
galactosyl donors 10, 11 under armed or disarmed conditions

| entry | donor | acceptor | time | | product | yield | α/β |
|---|---|---|---|---|---|---|---|
| 1^{†} | 10 | 4 | 30 | min | 14 | 95% | 51/49 |
| 2^{‡} | 10 | 12 | 1 | h | 15 | 65% | 2/1 |
| 3^{§} | 10 | 13 | 1 | h | 16 | <2% | N.A. |
| 4^{¥} | 11 | 12 | 1 | h | 15 | N.F. | N.A. |
| 5^{¥} | 11 | 13 | 1 | h | 16 | N.O. | N.A. |

^{†}NIS and TfOH (cat.) under 0° C. was used.
^{‡}NIS and TfOH (cat.) under −78° C.→−20° C. was used.
^{§}The presence of the products was confirmed by ESI-MS.
^{¥}TMSOTf and co-solvents: Et₂O/THF 5:1 under −23° C. was used.
N.A.: not available;
N.O.: not observed;
N.F.: not formed but only a silylated acceptor byproduct was obtained.

Although the concomitant reduction for both benzyl and azido groups of galactosyl sphingosine was difficult (Fan, G. T.; Pan, Y.-S; Lu, K.-C.; Cheng, Y.-P.; Lin, W.-C.; Lin, S.; Lin, C.-H.; Wong, C.-H.; Fan, J.-M.; Lin, C.-C. Tetrahedron, 2005, 61, 1855), compound 14 could be fully deprotected by using the reagent combination of H₂, MeOH/CHCl₃, AcOH and Pd(OH)₂. For example, the β-anomer 14β was used to test this condition and the deprotected product 17β could be obtained in 86% yield (Scheme 3).

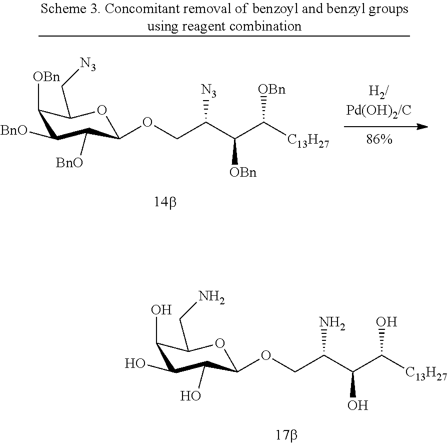

For comparing with the 18-carbon-based KRN7000, the galactosyl sphingosine 2 was used as another core compound (Scheme 4). Its preparation was relatively straightforward through a stepwise removal of both ester- and ether-protecting groups.

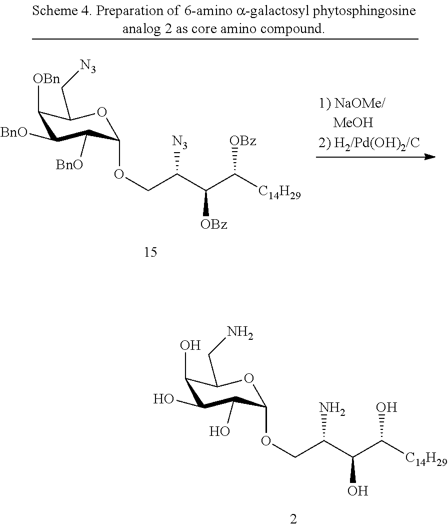

Since the more accessable core compound 1 was obtained in sufficient quantity, it was therefore adequate for further elaboration of amide products (Scheme 5) and screened for cytotoxicities.

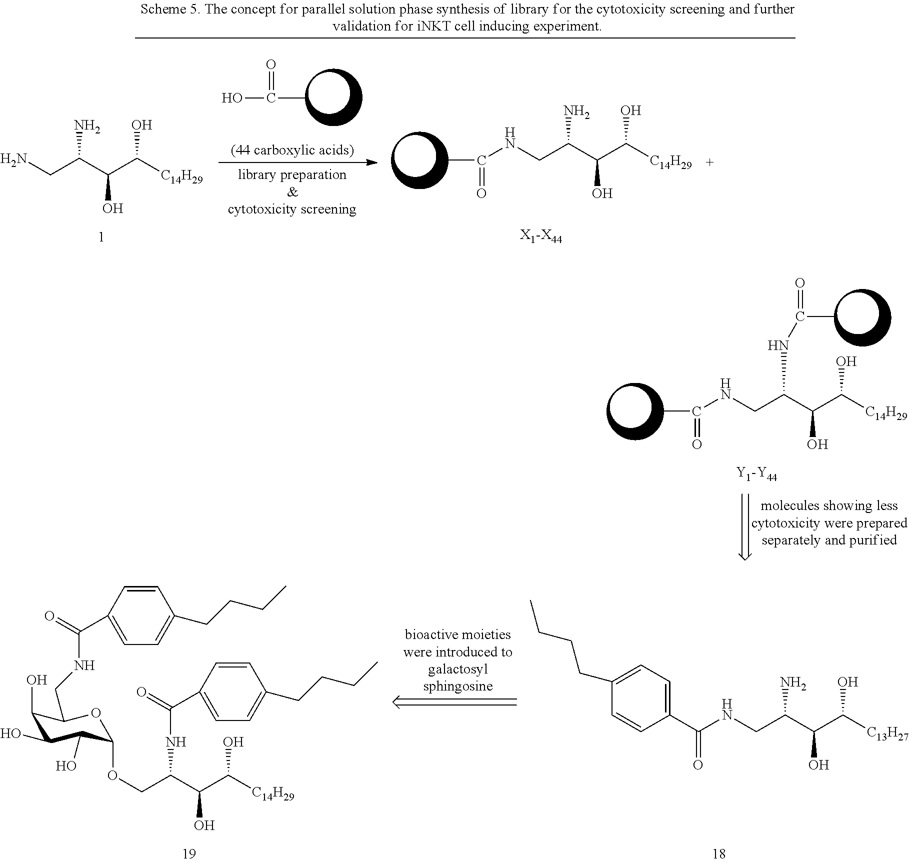

The subsequent preparation of library was starting from core 1 (20 mg) by coupling with 44 carboxylic acids using equivalent molarities. The initial screening for the cytotoxicities of these amide product mixtures was performed by using MTT assay with normal tissue derived fibroblast cells. Analog 18 showed the less cytotoxicites against normal human fibroblasts (50% cell viability vs. 0-5% of other analogs in U87 cells).

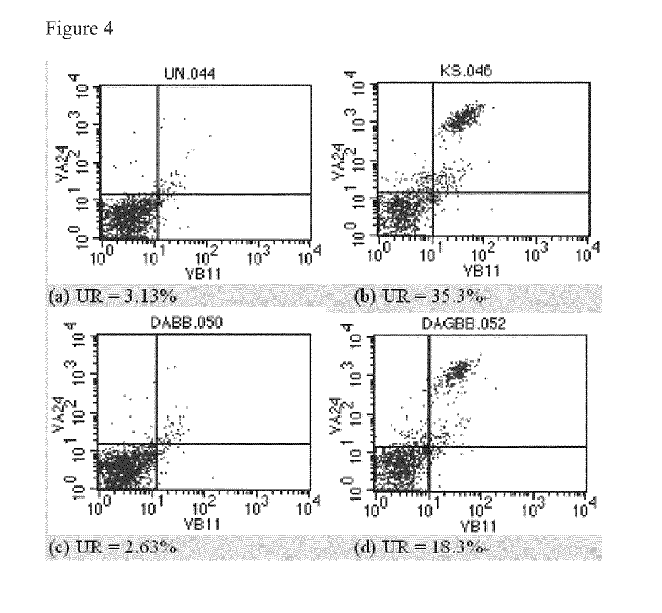

The less toxic product mixtures were further examined. These sample mixtures after simple filtration through silica gel were submitted to analysis with ESI-MS. Five product samples showed the expected molecular ion peak patterns, respectively. Among them, 4-benzyl benzoic acid derived amide product showing the most significant signals was resynthesized in both its ceramidic form 18 and galactosyl ceramidic form 19 (Scheme 6). The subsequent validation experiment was performed by MTT assay and flowcytotmetry (FIG. 4). Interestingly, a-GalCer analog 19 was Vα24+/Vβ11+iNKT cells stimulative but less cytotoxic compound 18 did not show an equivalent activity. This confirmed the important role played by sugar moieties.

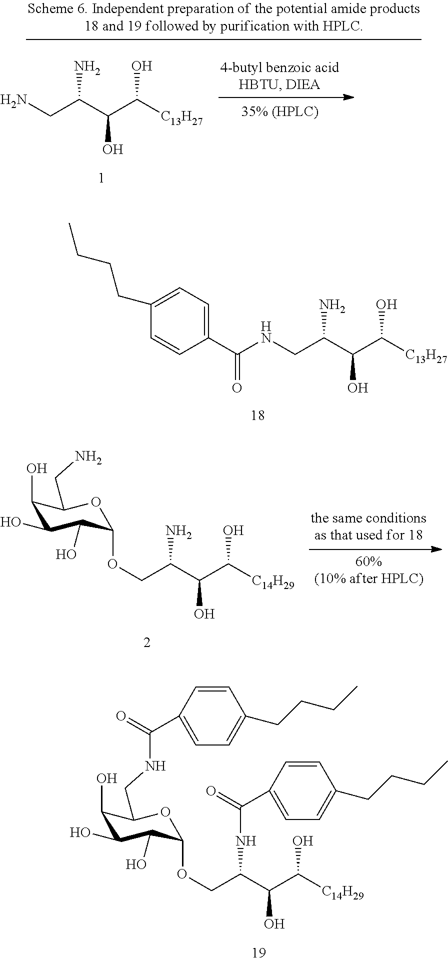

The preparation of 4-butyl-N-(((2R,3R,4S,5R,6S)-6-(((2S,3S,4R)-2-(4-butylbenzamido)-3,4-dihydroxyoctadecyl)oxy)-3,4,5-trihydroxytetrahydro-2H-py ran-2-yl)methyl)benzamide (19)

To a mixture of 4-butylbenzoic acid (23 mg, 0.13 mmol, 2.1 equiv), HBTU (57 mg, 0.15 mmol, 2.4 equiv) and DMF (6 mL) was added diisopropyl ethyl amine (14ᵢ1L, 0.08 mmol, 1.3 equiv) under N₂. After stirring for 10 min, TLC (EtOAc: n-hexane =1:3) indicated the formation of the ester intermediate (R_{f}=0.73) and consumption of the starting 4-butyl benzoic acid (R_{f}=0.12). To this mixture was added the solution of compound 2 (30 mg, 0.06 mmol) in DMF (4 mL). After stirring for 30 h, TLC (NH₃/MeOH/CHCl₃=0.2/1/1) indicated the formation of the product 19 (R_{f}=0.89) and consumption of the starting compound 2 (R_{f}=0.14). The mixture was concentrated under reduced pressure. The residue obtained was purified using column chromatography (EtOAc:n-hexane=1:4) to afford the white solid 19 in 60% yield (31 mg). The sample was further purified using HPLC (0.9 cm×20 cm, Si-100) with eluents MeOH/CHCl₃=1/29 at a flow rate of 3 mL/min to afford white solid (5 mg). t_{R}=19.2 min; t_{R}=11.9 min (aromatic impurities). Anal. C₄₆H₇₄N₂O₉, M (calcd.)=798.5 (m/z), ESI+Q−TOF: M=798.6 (m/z), [M+H]⁺=799.6 (19.04%), 800.6 (10.99%), [M+Na]⁺821.6 (100%), 822.6 (50.09%), 823.6 (11.33%), equivalent to the calculated isotopic ratio 100:50.8:12.7; HRMS (ESI) M (calcd.)=798.53943 (m/z), M (found)=798.53975 (m/z),¹H-NMR (500 MHz, CD₃OD): δ 0.87-0.94 (m, 9H, H_{aliphatic}), 1.21-1.40 (m, 28H, H_{aliphatic}), 1.60-1.70 (m, 5H, H_{aliphatic}), 2.61-2.67 (m, 4H), 3.44-3.48 (dd, 1H, J=7.5Hz), 3.56-3.60 (m, 1H), 3.67-3.82 (m, 6H), 3.93-4.00 (m, 2H), 4.40-4.44 (dd, 1H, J =10.5Hz, J =5.0Hz), 4.93-4.94 (d, 1H, J =3.5Hz, H₁), 7.20-7.25 (m, 4H, ArH), 7.67-7.71 (m, 4H, ArH); ¹³C-NMR(125 MHz, CD₃OD): δ 14.18 (CH₃), 14.36 (CH₃), 23.29 (CH₂), 23.32 (CH₂), 23. 67 (CH₂), 26.77 (CH₂), 30.41(CH₂), 30.71 (CH₂), 30.75 (CH₂), 33.03 (CH₂), 33.47 (CH₂), 34.54 (CH₂), 36.46 (CH₂), 41.69 (CH₂), 52.60 (CH₂), 67.96 (CH₂), 70.18 (CH), 70.52 (CH), 71.29 (CH), 71.46 (CH), 73.04 (CH), 75.85 (CH₃), 101.13 (CH), arom: 128.38, 128.46, 129.57, 132.93, 133.19, 148.34, 148.27; 169.93 (amide), 170.76 (amide).

One skilled in the art readily appreciates that the present invention is well adapted to carry out the objects and obtain the ends and advantages mentioned, as well as those inherent therein. The compounds, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A compound selected from compound of formula (I), pharmaceutically acceptable salts of compound of formula (I), and pharmaceutically acceptable prodrugs of compound of formula (I)

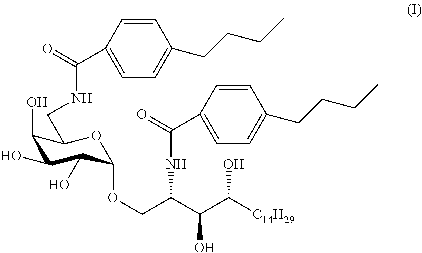

2. A composition comprising a compound of claim 1 and a pharmaceutically acceptable excipient.

3. The composition of claim 2, which activates natural killer T cells.

4. The composition of claim 3, which activates invariant natural killer T cells.

5. A method for preparing a compound that activates natural killer T cells, comprising:
establishing a library by coupling a compound of formula (II) with at least 44 carboxylic acids; 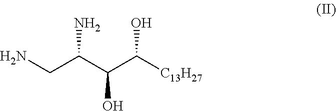
using MTT assay to screen for the cytotoxicities of these amide product mixtures of the library;
analyzing the amide product mixtures showing less cytotoxicities by ESI-MS;
wherein the amide product mixtures showing less cytotoxicities are the amide product mixtures with viability at least 10% higher than average viability in the MTT assay;
resynthesizing the amide product showing significant MS signals in its galactosyl ceramide form; and
validating the activity of the galactosyl ceramide by flowcytometry.

6. The method of claim 5, wherein the natural killer T cells are invariant natural killer T cells.
